(19) United States
(12) Patent Application Publication
(10) Pub. No.: US 2025/0169759 A1
(43) Pub. Date: May 29, 2025

(54) MULTI-SITE NONINVASIVE MEASUREMENT OF A PHYSIOLOGICAL PARAMETER

(71) Applicant: Masimo Corporation, Irvine, CA (US)

(72) Inventors: Vikrant Sharma, Santa Ana, CA (US); Philip Perea, Aliso Viejo, CA (US); Prashanth Iyengar, Irvine, CA (US)

(21) Appl. No.: 19/029,990

(22) Filed: Jan. 17, 2025

Related U.S. Application Data
(63) Continuation of application No. 18/048,365, filed on Oct. 20, 2022, now Pat. No. 12,232,888.
Application No. 18/048,365 is a continuation of application No. 15/824,983, filed on Nov. 28, 2017, now Pat. No. 11,504,058.
(60) Provisional application No. 62/429,458, filed on Dec. 2, 2016.

(51) Int. Cl. A61B 5/00; A61B 5/0205; A61B 5/024; A61B 5/145; A61B 5/1455

(52) U.S. Cl. CPC A61B 5/6814; A61B 5/0024; A61B 5/0205; A61B 5/742; A61B 5/0075; A61B 5/02416; A61B 5/14532; A61B 5/1455; A61B 5/6826; A61B 5/6829; A61B 2562/04

(57) ABSTRACT

A patient monitor can noninvasively measure a physiological parameter using sensor data from different measurement sites on a patient. The patient monitor can combine all sensor data from different measurement sites into a raw or minimally processed data form to generate a single, robust measurement of the physiological parameter. An optical sensor of a patient monitor can include multiple photodetectors each configured to generate a signal when detecting light attenuated by the patient's tissue. A measurement of a physiological parameter can be determined based on at least in part on the multiple signals from the multiple photodetectors.

### INCORPORATION BY REFERENCE TO ANY PRIORITY APPLICATIONS

The present application is a continuation of U.S. application Ser. No. 18/048,365, filed Oct. 20, 2022, titled “MULTI-SITE NONINVASIVE MEASUREMENT OF A PHYSIOLOGICAL PARAMETER”, which is a continuation of U.S. application Ser. No. 15/824,983, now U.S. Pat. No. 11,504,058, filed Nov. 28, 2017, titled “MULTI-SITE NONINVASIVE MEASUREMENT OF A PHYSIOLOGICAL PARAMETER”, which claims priority benefit under 35 U.S.C. § 119(e) to U.S. Provisional Patent Application Ser. No. 62/429,458, filed Dec. 2, 2016, titled “MULTI-SITE NONINVASIVE MEASUREMENT OF A PHYSIOLOGICAL PARAMETER”, all of which applications are hereby incorporated by reference in their entirety. Any and all applications for which a foreign or domestic priority claim is identified in the Application Data Sheet as filed with the present application are hereby incorporated by reference under 37 CFR 1.57.

### FIELD

The present disclosure relates to devices and methods for monitoring a patient's physiological information. More specifically, the present disclosure relates to noninvasive measurement of the physiological information using multiple sources of data.

### BACKGROUND

Hospitals, nursing homes, and other patient care facilities typically include patient monitoring devices in the facility. Patient monitoring devices can include sensors, processing equipment, and displays for obtaining and analyzing a medical patient's physiological parameters such as blood oxygen saturation level, respiratory rate, pulse, and a myriad of other parameters, such as those monitored on commercially available patient monitors from Masimo Corporation of Irvine, California. Clinicians, including doctors, nurses, and other medical personnel, use the physiological parameters and trends of those parameters obtained from patient monitors to diagnose illnesses and to prescribe treatments. Clinicians also use the physiological parameters to monitor patients during various clinical situations to determine whether to increase the level of medical care given to patients.

Patient monitoring can be achieved through spectroscopic analysis using, for example, a pulse oximeter. A pulse oximeter generally includes one or more light sources transmitting optical radiation into or reflecting off through a portion of the body, for example a digit such as a finger, a hand, a foot, a nose, an earlobe, or a forehead. One or more photo detection devices detect the light after attenuation by tissue and fluids of the portion of the body, and output one or more detector signals responsive to the detected attenuated light. One or more signal processing devices process the detector signals and output a measurement indicative of a blood constituent of interest, such as oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or otherwise, and the oximeter may display on one or more monitors the foregoing parameters individually, in groups, in trends, as combinations, or as an overall wellness or other index. An example of such an oximeter is described in U.S. application Ser. No. 09/323,176, filed May 27, 1999, titled “Stereo Pulse Oximeter,” now U.S. Pat. No. 6,334,065, the disclosure of which is hereby incorporated by reference in its entirety.

In noninvasive devices and methods, a sensor is often adapted to position the portion of the body proximate the light source and light detector. Some noninvasive sensors can include a finger clip that includes a contoured bed conforming generally to the shape of a finger. An example of such a noninvasive sensor is described in U.S. application Ser. No. 12/829,352, filed Jul. 1, 2010, titled “Multi-Stream Data Collection System for Noninvasive Measurement of Blood Constituents,” now U.S. Pat. No. 9,277,880, the disclosure of which is hereby incorporated by reference in its entirety. Some noninvasive sensors can include one or more sensing components, such as the light source and/or the photodetectors on an adhesive tape, such as described in U.S. application Ser. No. 13/041,803, filed May 7, 2011, titled “Reprocessing of a physiological sensor,” now U.S. Pat. No. 8,584,345, the disclosure of which is hereby incorporated by reference in its entirety.

### SUMMARY

Conventional physiological monitors, such as pulse oximeters, typically are restricted to measuring physiological information at a single patient site. Stereo pulse oximeter as described in U.S. Pat. No. 6,334,065, can receive data input from sensors placed at multiple measurement sites of a patient and output simultaneous or substantially simultaneous measurements at each of the measurement sites. However, a processor of the stereo pulse oximeter as described in U.S. Pat. No. 6,334,065 processes data input from sensors placed at each measurement site independent of data from sensors at the other measurement sites. Useful information can be obtained if raw or minimally processed sensor data from different measurement sites can be combined and fed into the processor to calculate a single measurement of a physiological parameter. Minimally processed sensor data can be demodulated, filtered, and/or otherwise processed to prepare the data for use by the signal processor to calculate measurements of physiological parameters. This disclosure describes embodiments of noninvasive methods and devices for processing data input from optical sensors located at multiple measurement sites to generate a single measurement of a physiological parameter. The parameter can be a measurement of blood constituent or analyte, such as oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or of many other physiologically relevant patient characteristics.

Sensors with multiple photodetectors can be used in an optical sensor to also provide useful information about the patient's physiological condition and/or the placement of the optical sensor. Each of the photodetectors can provide a respective output stream based on the detected optical radiation, or a sum of output streams can be provided from multiple photodetectors. An example of a sensor which employs multiple photodetectors is described in U.S. Pat. No. 9,277,880. Configurations of those multiple photodetectors can include a substantially linear geometry or substantially two-dimensional grid geometry. However, the anatomy at most measurement sites is three-dimensional and irregularly-shaped. The optical sensor with multiple detectors disclosed herein has an improved spatial configuration. The multiple photodetectors described herein can be placed on various locations on a three-dimensional contour of the measurement site on the patient. Use of multiple photodetectors in a spatial configuration can have several advantages. The multiple photodetectors can allow a caregiver or a patient to confirm or validate that the sensor is positioned correctly. This is because the multiple locations of the spatial configuration can provide, for example, topology information that indicates where the sensor has been positioned. This spatial configuration can also provide a diversity of light path lengths among at least some of the detectors and improve robustness of measurements of the physiological parameter(s). The optical sensor described herein can have multiple light emitters and/or multiple photodetectors.

A noninvasive physiological monitor can include at least two sensor interfaces each in communication with at least one sensor adapted to be positioned at one of at least two measurement sites of a patient, each of the interfaces having an output responsive to light transmitted through one of the at least two measurement sites. The system can also include a signal processor in communication with the at least two interface outputs, the signal processor configured to combine the interface outputs to generate a single measurement of physiological parameters. Data from the sensors located at multiple measurement sites can be fed into a single algorithm in the signal processor. The sensors can each include a plurality of light emitters configured to emit optical radiation onto a tissue of one of the at least two measurement sites.

A method of calculating a single measurement of physiological parameters can include transmitting light of at least first and second wavelengths through tissues of at least two measurement sites of a patient; detecting the light attenuated by the tissues of the at least two measurement sites; generating a signal output based on the light detected at each of the at least two measurement sites; and determining the single measurement of the physiological parameters by combining the signal outputs relating to the at least two measurement sites.

A noninvasive sensor capable of producing a signal responsive to light attenuated by tissue at a measurement site on a patient can include an optical source configured to emit optical radiation onto the tissue at the measurement site. The sensor can also include more than one photodetector configured to detect the optical radiation form the optical source after attenuation by the tissue of the patient and to output more than one respective signal stream responsive to the detected optical radiation. When the sensor is coupled to the measurement site, the photodetectors can be distributed throughout a sensor area in contact with the measurement site. The photodetectors can be arranged in a three-dimensional configuration conforming to a contour of the measurement site when the sensor is attached to the measurement site. The sensor can further include a housing for positioning the optical source and the more than one photodetectors with respect to the measurement site, or an adhesive tape portion for positioning the optical source and the more than one photodetectors with respect to the measurement site.

A method of measuring a physiological parameter using multiple photodetectors in one sensor can comprise transmitting light from a light source through tissue of a measurement site of a patient; detecting the light after the light has passed through the tissue of the measurement site; generating a data stream based on the light detected by each of a plurality of photodetectors, the plurality of photodetectors distributed throughout a sensor area in contact with the measurement site; and determining the physiological parameter from the data streams of the plurality of photodetectors.

A noninvasive physiological monitor can include at least two optical sensors, at least two sensor interfaces each in communication with one of the at least two sensors, and a signal processor. Each sensor can have a plurality of light emitters configured to emit optical radiation onto tissue of one of at least two measurement sites of a patient and a plurality of photodetectors configured to detect the optical radiation attenuated by the tissue of the one of at least two measurement sites, the plurality of photodetectors distributed throughout a sensor area in contact with the one of the at least two measurement sites. Each of the interfaces can have an output responsive to the optical radiation attenuated by the tissue of the one of at least two measurement sites. The signal processor can be in communication with the at least two interface outputs and configured to combine the interface outputs to generate a single measurement of physiological parameters.

A noninvasive physiological monitor can comprise at least one input in communication with first and second sensors adapted to be respectively positioned at first and second measurement sites of a patient, the at least one input configured to receive one or more signals responsive to light of at least a plurality of wavelengths attenuated by tissue at the first and second measurement sites of the patient, the one or more signals outputted by one or more optical detectors of the first and second sensors; and one or more signal processors configured to combine the one or more signals from the first and second sensors into combined sensor data and to generate a single measurement of a physiological parameter based on the combined sensor data. The first and second measurement sites can comprise different types of body tissues. The first measurement site can be on or around the patient's head. The first measurement site can be the patient's nose. The second measurement site can be a peripheral site of the patient. The second measurement site can be the patient's finger. The first and/or second sensor can comprise a plurality of light emitters configured to emit optical radiation onto the tissue of the first and/or second measurement sites. The first and/or second sensor can comprise a plurality of light detectors configured to detect light attenuated by the tissue of the first and/or second measurement sites. The physiological parameter can comprise oxygen saturation, pulse rate, a plethysmograph waveform, perfusion index, pleth variability index, methemoglobin, carboxyhemoglobin, total hemoglobin, and/or glucose.

A method of calculating a single measurement of a physiological parameter can comprise using a first sensor including one or more light emitters, emitting light of at least first and second wavelengths into tissues of a first measurement site of a patient; using one or more electronic light detectors of the first sensor, detecting the light attenuated by the tissues of the first measurement site and outputting at least a first signal based on the light detected at the first measurement site; using a second sensor including one or more light emitters, emitting light of at least first and second wavelengths into tissues of a second measurement site of a patient, the second measurement site different from the first measurement site; using one or more electronic light detectors of the first sensor, detecting the light attenuated by the tissues of the second measurement site and outputting at least a second signal based on the light detected at the second measurement site; and using one or more signal processors, combining the at least a first signal and the at least a second signal into combined sensor data, and processing the combined sensor data to determine the single measurement of the physiological parameter. The first and second measurement sites can comprise different types of body tissues. The first measurement site can be on or around the patient's head. The first measurement site can be the patient's nose. The second measurement site can be a peripheral site of the patient. The second measurement site can be the patient's finger. The first and/or second sensor can comprise a plurality of light emitters configured to emit optical radiation onto the tissue of the first and/or second measurement sites. The first and/or second sensor can comprise a plurality of light detectors configured to detect light attenuated by the tissue of the first and/or second measurement sites. The physiological parameter can comprise oxygen saturation, pulse rate, a plethysmograph waveform, perfusion index, pleth variability index, methemoglobin, carboxyhemoglobin, total hemoglobin, and/or glucose.

A noninvasive physiological monitor can comprise a first optical sensor having one or more light emitters configured to emit optical radiation onto tissue of a first measurement site of a patient and one or more of photodetectors configured to detect the optical radiation attenuated by the tissue of the first measurement site and to output a signal responsive to the light attenuation by the tissue of the first measurement site; a second optical sensor having one or more light emitters configured to emit optical radiation onto tissue of a second measurement site of the patient and one or more of photodetectors configured to detect the optical radiation attenuated by the tissue of the second measurement site and to output a signal responsive to the light attenuation by the tissue of the second measurement site; and one or more signal processors in communication with the first and second optical sensors, the signal processor configured to combine the signals responsive to the light attenuation by the tissues of the first and second measurement sites into combined sensor data, and to generate a single measurement of a physiological parameter from the combined sensor data. The one or more photodetectors can be distributed throughout a sensor area in contact with one of the first and second measurement sites. The first and second measurement sites can comprise different types of body tissues. The first measurement site can be on or around the patient's head. The first measurement site can be the patient's nose. The second measurement site can be a peripheral site of the patient. The second measurement site can be the patient's finger. The physiological parameter can comprise oxygen saturation, pulse rate, a plethysmograph waveform, perfusion index, pleth variability index, methemoglobin, carboxyhemoglobin, total hemoglobin, and/or glucose.

For purposes of summarizing the disclosure, certain aspects, advantages and novel features of the inventions have been described herein. It is to be understood that not necessarily all such advantages can be achieved in accordance with any particular embodiment of the inventions disclosed herein. Thus, the inventions disclosed herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

Various embodiments will be described hereinafter with reference to the accompanying drawings. These embodiments are illustrated and described by example only, and are not intended to limit the scope of the disclosure. In the drawings, similar elements have similar reference numerals.

FIG. 1A illustrates an example block diagram of an optical-based multi-site physiology monitor.

FIG. 1B illustrates an example block diagram of an optical-based multi-site physiology monitor.

FIG. 1C illustrates an example block diagram of an optical-based multi-site physiology monitor.

FIG. 2 illustrates an example block diagram of the sensors coupled to the physiological monitor in FIGS. 1A-1C.

FIG. 3 illustrates an example optical sensor of a physiological monitor.

FIG. 4 illustrates an example block diagram of a physiological monitor employing the sensor of FIG. 3.

FIG. 5 illustrates a block diagram of an example optical sensor of a multi-site physiological monitor.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

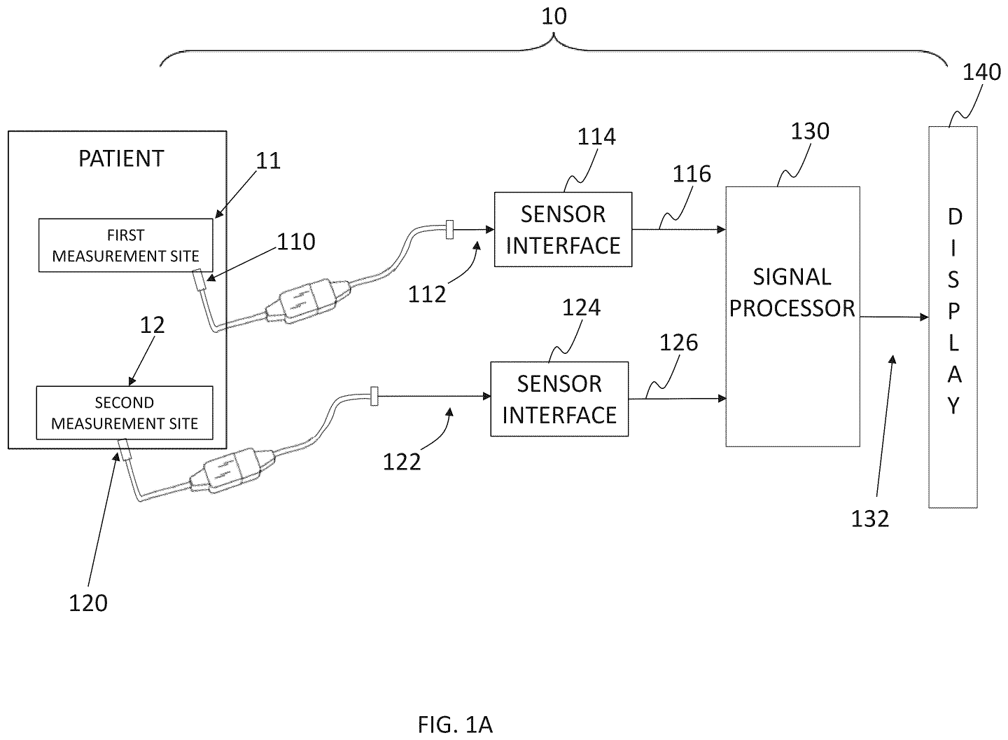

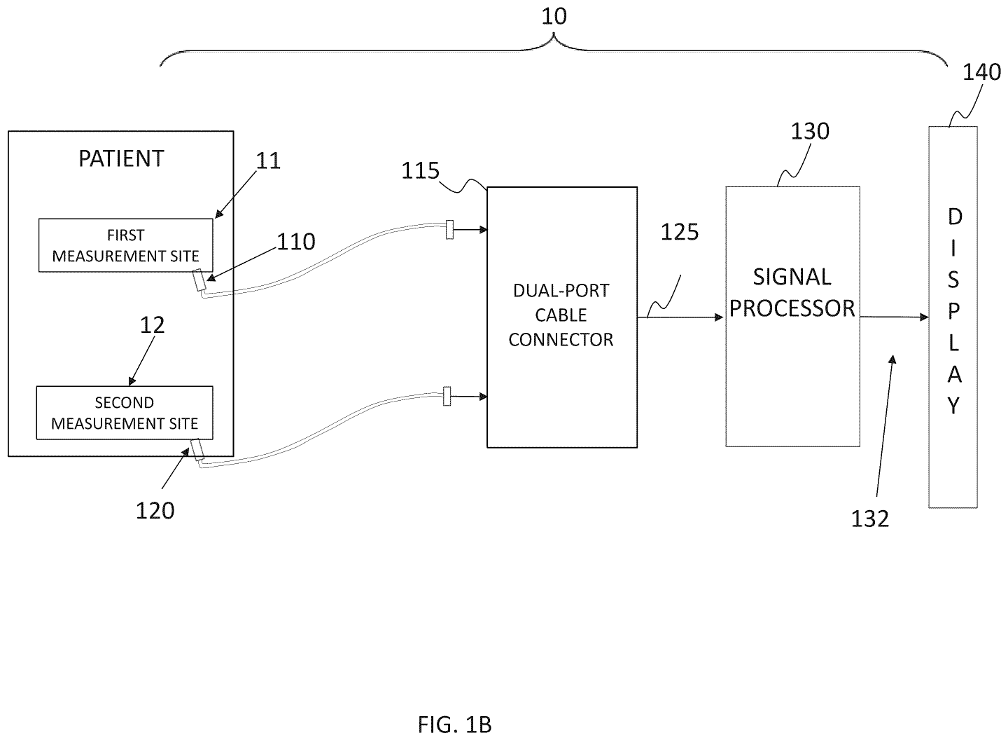

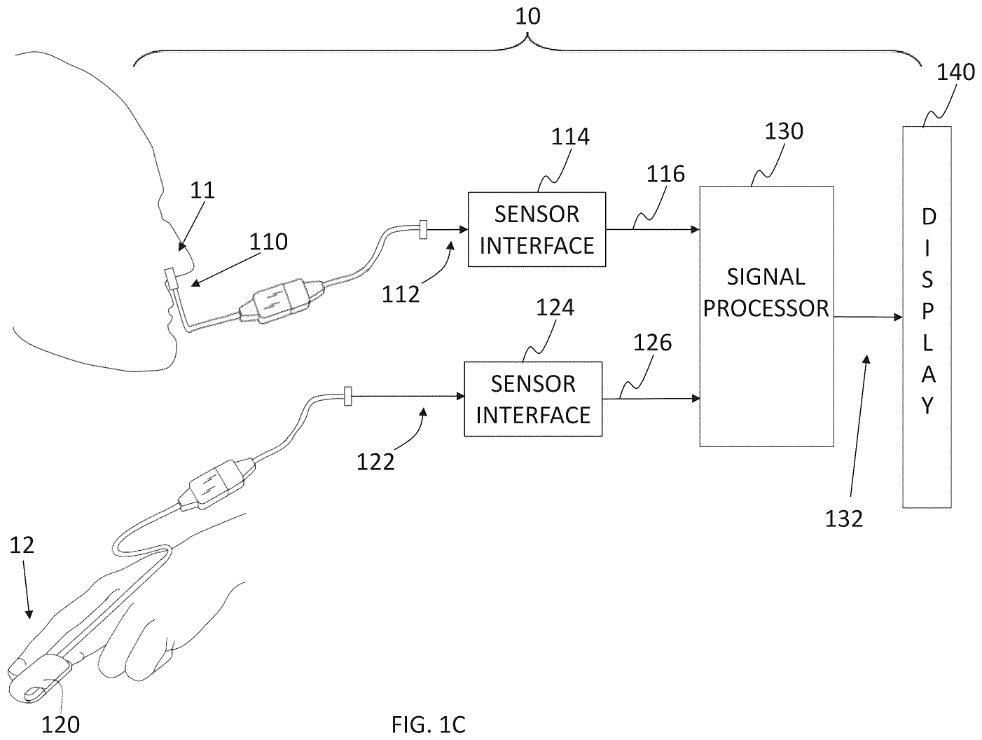

FIGS. 1A-1B illustrate an example optical-based physiological monitor 10. The monitor 10 is configured for processing data from optical sensors 110, 120 located at multiple patient measurement sites 11, 12 to generate a single noninvasive measurement of a physiological parameter.

As shown in FIGS. 1A-1B, the monitor 10 can include a first optical sensor 110 attached to a first measurement site 11 and a second sensor 120 attached to a second measurement site 12 of a patient. The first measurement site 11 can be a cephalic measurement site, which can be on or around the patient's head. Examples of the cephalic measurement site can include the patient's nose, earlobe, or others. The second measurement site 12 can be a peripheral measurement site, which can be located on or near the patient's limbs. Examples of the peripheral measurement site can include the patient's arm, palm, fingers, leg, ankle, foot, toes, or others. Sensor data responsive to the light attenuation at each measurement site can provide information related to the patient's blood constitute and/or analyte that may not be available from a single site measurement, and/or allow cross-checking between the data from both measurement sites to improve accuracy and robustness of the measurements. Combining the sensor data from each measurement site can also provide the signal processor with more data from which to obtain measurements of one or more physiological parameter.

FIG. 1C illustrates an example application of the monitor 10 of FIGS. 1A-1B. As shown, the first measurement site 11 can be the patient's nose and the second measurement site 12 can be the patient's fingertip. The number of sensors and the location of measurement sites are not limiting. For example and not by way of limitation, three, four, or more sensors may respectively be attached to any of the patient's finger, hand, foot, and/or face. The first and/or second measurement sites 11, 12 can also comprise other locations of the patient's body. For example, the first and/or second measurement sites 11, 12 can comprise two cephalic measurement sites, two peripheral measurement sites, or any two measurement sites that comprise different types of body tissues. The physiological monitor 10 can be applied on an adult patient and/or a neonatal patient.

Each sensor 110, 120 can provide a stream of data to a signal processor 130. For example, as shown in FIG. 1A, the first sensor 110 can be connected to an input 112 of a first sensor interface 114 via a first cable connector 111. An output 116 of the first sensor interface 114 can be fed into the signal processor 130 in a raw or minimally processed data form. Similarly, the second sensor 120 can be connected to an input 122 of a second sensor interface 124 via a second cable connector 121. An output 126 of the second sensor interface 124 can also be fed into the signal processor 130 in a raw or minimally processed data form. Minimally processed sensor data can be demodulated, filtered, and/or otherwise processed to prepare the data for use by the signal processor to calculate measurements of physiological parameters. Minimally processing the sensor data can also optionally include generating basic ratios of wavelengths in a multi-wavelength system. As shown in FIG. 1B, the first and second sensors 110, 120 can be coupled to a dual-port cable connector 115, which can output signals 125 of the first and second sensors in raw data form or minimally processed form. An example dual-port cable connector can be a duo connector described in U.S. Pat. No. 8,315,683, filed Sep. 20, 2007, the entirety of which is incorporated herein by reference. The signal processor 130 can comprise one or more hardware and/or software signal processors. Minimal processing can include, for example, a low or high pass filter or other preprocessing steps typically performed in a signal acquisition system.

The unprocessed or minimally processed streams of data from the first and second sensors 110, 120 can be combined by the signal processor 130 into combined sensor data. The combined sensor data can include a plurality of features of the signals from the first and second sensors 110, 120, such as amplitude, phase, DC value, or others. The measurement site and/or body tissue information can be provided to the signal processor 130 as independent features. The signal processor 130 can produce a single measurement 132 of a physiological parameter based on the combined sensor data. The plurality of features of the signals can be mapped onto empirical data, which can provide estimates of physiological parameter measurements. The additional independent features of the measurement site and/or body tissue information can provide more combinations of features and improve the estimation of the physiological parameters. The combinations of features can be linear and/or can include high-ordered combinations. The signal processor 130 can analyze from the combined sensor data various features, such as a ratiometric value, such as ratios of attenuated light of one wavelength to attenuated light of another wavelength, and/or combinations of features, including non-normalized features, data from bulk absorption and/or peripheral absorption signals, or others.

The single measurement 132 can be displayed on a display 140 of the monitor 10. The measured physiological parameter can include one or more of oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or of many other physiologically relevant patient characteristics. The signal processor 130 can also optionally process the combined data from the first and second sensors 110, 120 using a single algorithm. The single measurement 132 can provide caregivers with the patient's systematic status of the physiological information. The single measurement 132 from the combined sensor data may be more informative of the well-being of the patient than measurements from a single patient site. Combining streams of data from multiple patient sites can also reduce errors and/or noise associated with a particular sensor to provide a more robust reading of the physiological parameter.

The signal processor 130 of the monitor 10 can process streams of data from the sensors into combined sensor data to generate a single measurement, and/or to independently generate measurements for each of the measurement sites.

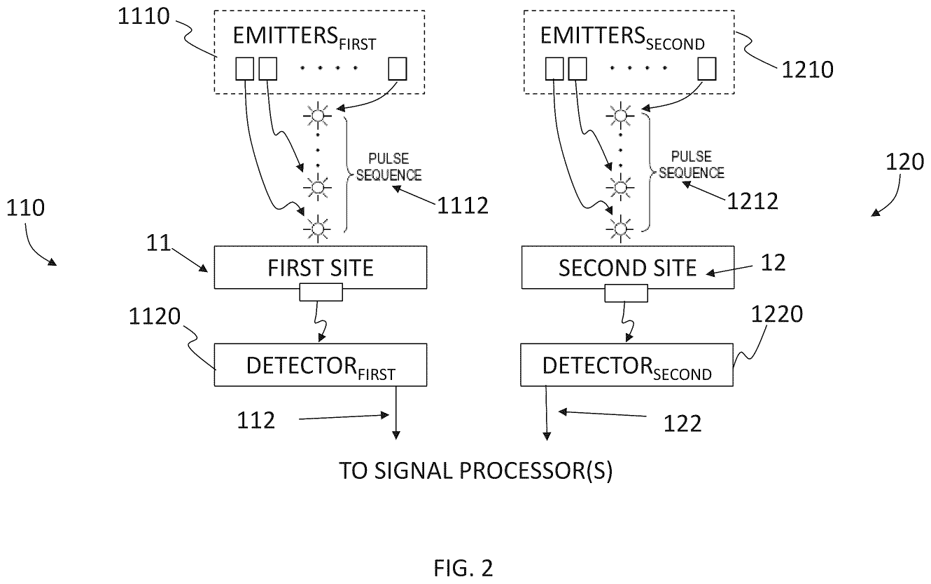

FIG. 2 illustrates example sensors that can be used with the monitor 10 of FIGS. 1A-1C. As shown in FIG. 2, the first sensor 110 can include a plurality of light emitters 1110. For example, the light emitters can be light-emitting diodes (LED). The light emitters 1110 can each emit light of a certain wavelength. The light emitters 1110 can also emit light of different wavelengths in sequence with only one emitter emitting light at a given time, thereby forming a pulse sequence 1112. The number of emitters is not limiting and can range from two to eight in each of the first and second sensors 110, 120. The second sensor 120 can have a plurality of emitters 1210 functioning in the same or different manner as the emitters 1110 of the first sensor 110. The second sensor 120 can have the same or different number of light emitters as the first sensor 110. For example, each the first and second sensors 110, 120 can have eight light emitters, or the first sensor 110 can have four emitters while the second sensor can have eight emitters. The emitters 1210 of the second sensor 120 can emit light of the same or different wavelengths as the emitters 1110 of the first sensor 110. The plurality of emitters 1210 of the second sensor 120 can generate a pulse sequence 1212. Detailed descriptions and additional examples of the light emitters are provided in U.S. Pat. No. 9,277,880.

With continued reference to FIG. 2, a photodetector 1120 of the first sensor 110 can detect the light from the emitters 1110 after the light is attenuated by tissue of the first measurement site 11. As shown, the first measurement site can be a cephalic site. The photodetector 1120 can comprise photodiodes, phototransistors, or the like. Details of the photodetector are described in U.S. Pat. No. 9,277,880. The photodetector 1120 can generate an electrical signal based on the detected light from the plurality of emitters 1110. A photodetector 1220 of the second sensor 120 can detect the light from the emitters 1210 after the light is attenuated by tissue of the second measurement site 12. As shown, the second measurement site can be a peripheral site. The photodetector 1220 can also generate an electrical signal based on the detected light from the plurality of emitters 1210. The signals of the first and second sensors 110, 120 can be provided to the signal processor for processing and determining measurements of physiological parameters.

The signal of the detected light from the plurality of emitters 1110 and the signal of the detected light from the plurality of emitters 1210 can be fed into the signal processor 130 such that the signal processor 130 can combine the two signals to process data from all the emitters 1110, 1210 into combined sensor data. For example, the signal processor 130 can process raw or minimally processed data from twelve to sixteen light emitters to generate a single measurement of physiological parameters. Having data from twelve to sixteen emitters can advantageously allow the processor to produce a more robust measurement. The signal processor 130 can additionally and/or alternatively process the output 116 of the first sensor interface 114 independently of the output 126 of the second sensor interface 124 to produce two measurements of the physiological parameter respectively. The signal processor 130 can determine a final measurement of the physiological parameter based on one or more of the single measurement, or the two measurements from the signals of the first and second sensors respectively. The final measurement can be an average or weighted average of those measurements, or others. The signal processor can also compare the single measurement from the combined data with the measurements from the individual sensors to obtain additional information about the patient.

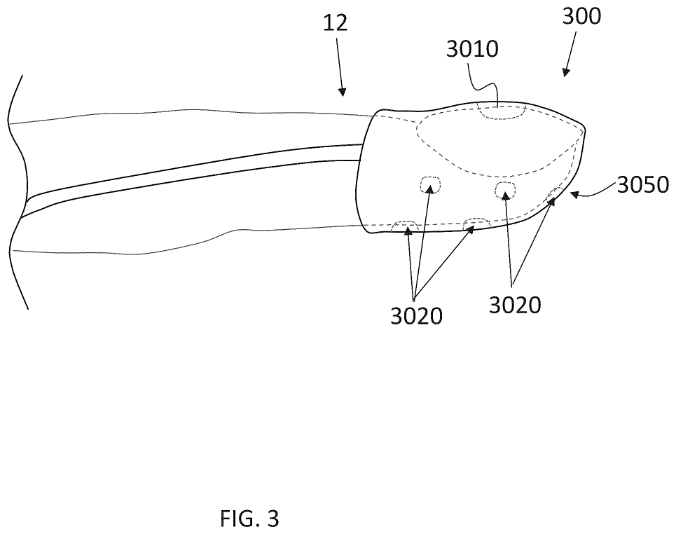

Turning to FIG. 3, an optical sensor 300 configured for use in an optical-based physiological monitor is disclosed. Although FIG. 3 illustrates attachment of the sensor 300 to the patient's fingertip 12, the sensor 300 can have a shape configured for attaching to other measurement sites, for example, the patient's hand, foot, or face including not but limited to the earlobe, the nose, and/or the forehead. The sensor 300 can have a housing with a predefined shape, for example and not by way of limitation, a finger clip configured for attaching to the patient's fingertip. The sensor 300 can also comprise a flexible tape having a sensor portion and an adhesive tape portion so as to conform to the patient's measurement site anatomy.

As shown in FIG. 3, the sensor 300 can have a light source 3010. The light source 3010 can comprise two or more light emitters. Each light emitter can be configured to emit light of a different wavelength. The sensor 300 can also have a plurality of photodetectors 3020. The number of photodetectors on the sensor 300 is not limiting and can range from two to twelve or more. The photodetectors 3020 can be arranged in a three-dimensional configuration. The photodetectors 3020 can be arranged with random spacing so that the detectors 3020 can be distributed throughout a contact area 3050 of the sensor. The photodetectors 3020 can have a spatial configuration conforming to a contour or patient anatomy of the measurement site. For a -clip type of sensor, the light source 3010 can be located in an upper portion of the clip and the photodetectors 3020 can be located in a lower portion of the clip. The photodetectors 3020 can be distributed throughout a contoured bed of the lower portion conforming generally to the patient anatomy of the measurement site.

For a tape type of sensor, the light source 3010 and the detectors 3020 can be arranged on a flat or substantially tape such that when the sensor 300 is taped to the measurement site, such as the fingertip 12, the light source 3010 can be spaced away from the plurality of photodetectors 3020 and the photodetectors 3020 can be at various locations of the fingertip 12. As shown in FIG. 3, the light source 3010 can be located near a center of the patient's finger nail and the light detectors 3020 can be distributed throughout the contact area 3050 of the sensor. Some of the photodetectors 3020 can be on an opposite side of the fingertip 12 from the light source 3010. Some of the photodetectors 3020 can be on medial and/or lateral sides of the fingertip 12. Light emitted by the light source 3010 can travel through different tissue depths and/or vasculature before reaching the plurality of photodetectors 3020.

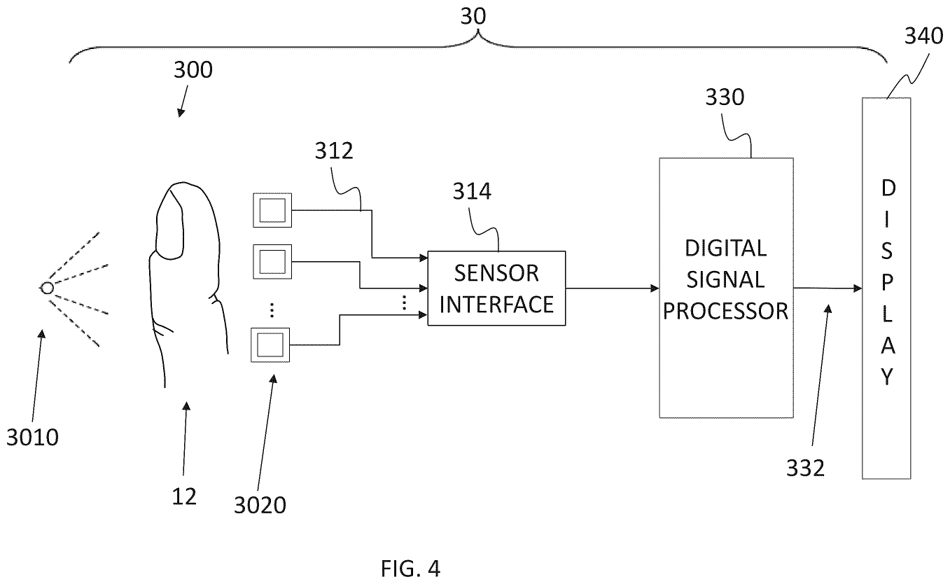

FIG. 4 illustrates an example optical-based physiological monitor 30 utilizing the sensor 300. The physiological monitor 30 can have the same or similar features as the physiological monitor 10 of FIGS. 1A-1C. Accordingly, features of the physiological monitors 10, 30 of FIGS. 1A-1C and 4 can be incorporated into one another. As shown in FIG. 4, signals 312 generated by all or substantially all of the photodetectors 3020 upon detecting the light attenuated by patient's finger tissue can be inputted through a sensor interface 314 to a signal processor 330. Details of processing signals from multiple detectors of a sensor are provided in U.S. Pat. No. 9,277,880. The signal processor 330 can generate measurements 332 of one or more physiological parameters from the multiple signals. The measurements 332 can be displayed on a display 340 of the pulse oximeter 30.

The measurements 332 can provide the caregiver with the physiological information of the entire tissue bed of the patient covered by the contact area 3050. The three-dimensional geometry of the distribution of the detectors 3020 on the fingertip 12 can provide a diversity of light paths among at least some of the photodetectors and improve robustness of the physiological parameter measurements. The signals from the multiple photodetectors 3020 can also inform the caregiver whether the sensor is positioned correctly. This is because the randomly-spaced photodetectors 3020 can provide, for example, topology information, which can indicate where the sensor has been positioned and/or whether the sensor has been positioned correctly.

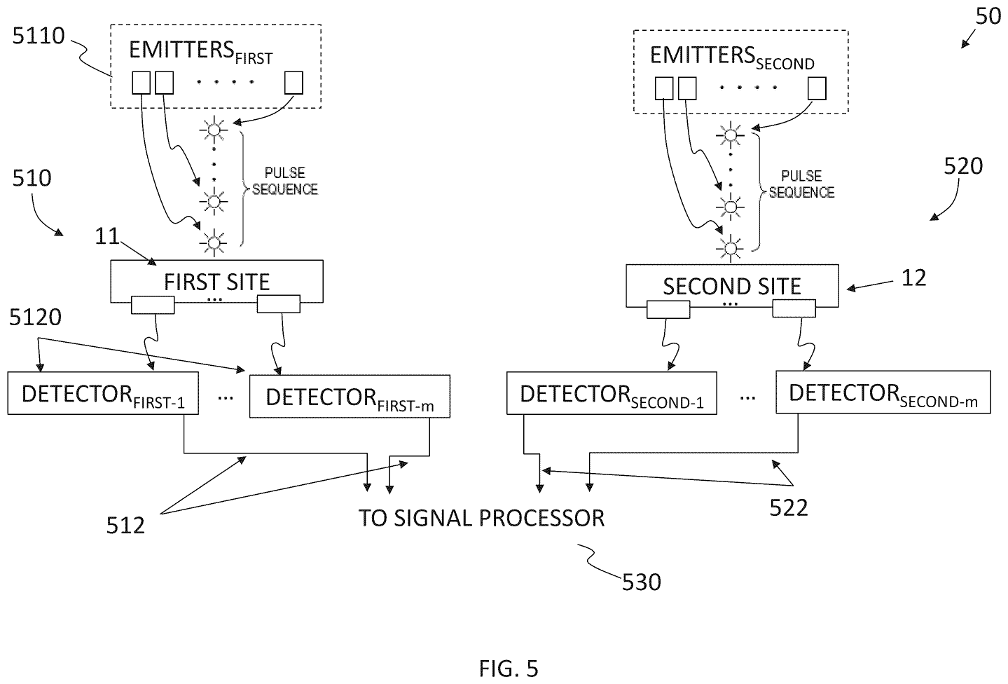

In FIG. 5, an example physiological monitor 50 with sensors 510, 520 connected to multiple patient sites 11, 12 is shown. The physiological monitor 50 can have the same or similar features as the physiological monitor 10 of FIGS. 1A-1C and/or the physiological monitor 30 of FIG. 4. Accordingly, features of the physiological monitors 10, 30, 50 of FIGS. 1A-1C, 4, and 5 can be incorporated into one another.

As shown in FIG. 5, the monitor 50 can have a first sensor 510 attached to a first measurement site 11 and a second sensor 520 attached to a second measurement site 12. The first measurement site 11 can be a cephalic site described above. The second measurement site 12 can be a peripheral site described above.

The first and second sensors 510, 520 can each have a plurality of light emitters 5110, 5210 and a plurality of photodetectors 5120, 5220. The number and/or spatial configuration of light emitters 5110, 5210 and/or photodetectors 5120, 5220 in each of the first and second sensors 510, 520 can be the same or different. When the first sensor 510 is attached to the first measurement site 11, the plurality of detectors 5120 can be distributed in a spatial configuration conforming to a shape of the patient anatomy at the first measurement site 11. When the second sensor 520 is attached to the second measurement site 12, the photodetectors 5220 can be distributed in a spatial configuration to a shape of the patient anatomy at the second measurement site 12. An example of the spatial configuration of the photodetectors 3020 is shown in FIG. 3.

The photodetectors 5120, 5220 can detect light emitted by at least some or all of the light emitters 5110, 5210 and can each generate a signal responsive to the detected light. Signals 512 from the photodetectors 5120 and signals 522 from the photodetectors 5220 can be provided to a signal processor 530 for processing into measurements of physiological parameters. The signal processor 530 can combine all the signals 512, 522 representing respective streams of data from the photodetectors based on light from at least some of the light emitters at each measurement site into combined sensor data. The signal processor 530 can generate a single measurement of the physiological parameter based on the combined sensor data. The signal processor 530 can also independently process the signals 512, 522 to generate measurements of the physiological parameter for each of the measurement sites.

The physiological monitor 50 can combine the advantages of the physiological monitors 10 and 30 of FIGS. 1-4 described above. The physiological monitor 50 can improve robustness of measurements of physiological parameters, can process a plurality of features in the combined signals, such as non-normalized signal features, which may not be possible or accurate when obtained from a single site measurement, and/or can provide topological information that can indicate, for example, whether the sensor has been correctly positioned.

Although this disclosure has been disclosed in the context of certain preferred embodiments and examples, it will be understood by those skilled in the art that the present disclosure extends beyond the specifically disclosed embodiments to other alternative embodiments and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. Accordingly, it should be understood that various features and aspects of the disclosed embodiments can be combined with or substituted for one another in order to form varying modes of the disclosed.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. (canceled)

2. A noninvasive physiological monitor, comprising:
a first sensor interface in communication with a first sensor adapted to be positioned at a first measurement site of a patient, the first sensor interface configured to (i) receive first one or more signals responsive to light of at least a plurality of wavelengths attenuated by tissue at the first measurement site, the first one or more signals outputted by one or more optical detectors of the first sensor, and (ii) output first one or more output detector signals responsive to the first one or more signals;
a second sensor interface in communication with a second sensor adapted to be positioned at a second measurement site of the patient, the second measurement site different from the first measurement site, the second sensor interface configured to (i) receive second one or more signals responsive to light of at least a plurality of wavelengths attenuated by tissue at the second measurement site, the second one or more signals outputted by one or more optical detectors of the second sensor, and (ii) output second one or more output detector signals responsive to the second one or more signals; and
one or more signal processors configured to receive the first and second one or more output detector signals and combine the first and second one or more output detector signals into combined data prior to feeding the combined data into a single algorithm to generate a single measurement of a physiological parameter.

3. The noninvasive physiological monitor of claim 2, wherein the first or second one or more output detector signals comprising one or more of raw, demodulated, filtered, or converted-to-digital data.

4. The noninvasive physiological monitor of claim 2, wherein the one or more signal processors are further configured to map the combined data to empirical data to generate the single measurement.

5. The noninvasive physiological monitor of claim 2, wherein the first and second measurement sites comprise different types of body tissues.

6. The noninvasive physiological monitor of claim 2, wherein the first measurement site is on or around the patient's head or the patient's nose.

7. The noninvasive physiological monitor of claim 2, wherein the second measurement site is a peripheral site of the patient.

8. The noninvasive physiological monitor of claim 7, wherein the second measurement site is the patient's finger.

9. The noninvasive physiological monitor of claim 2, wherein the first and/or second sensor comprises a plurality of light emitters configured to emit optical radiation onto the tissue of the first and/or second measurement sites.

10. The noninvasive physiological monitor of claim 9, wherein the first and/or second sensor comprises a plurality of light detectors configured to detect light attenuated by the tissue of the first and/or second measurement sites.

11. The noninvasive physiological monitor of claim 2, wherein the physiological parameter comprises oxygen saturation, methemoglobin, carboxyhemoglobin, total hemoglobin, and/or glucose.

12. The noninvasive physiological monitor of claim 2, wherein the combined data comprises one or more of a ratiometric value, non-normalized features, data from bulk absorption, or peripheral absorption signals.

13. A noninvasive physiological monitoring system, the system comprising:
a first optical sensor having one or more light emitters configured to emit optical radiation onto tissue of a first measurement site of a patient and one or more of photodetectors configured to detect the optical radiation attenuated by the tissue of the first measurement site and to output a first detector signal responsive to the light attenuation by the tissue of the first measurement site;
a second optical sensor having one or more light emitters configured to emit optical radiation onto tissue of a second measurement site of the patient and one or more of photodetectors configured to detect the optical radiation attenuated by the tissue of the second measurement site and to output a second detector signal responsive to the light attenuation by the tissue of the second measurement site; and
one or more signal processors in communication with the first and second optical sensors, the one or more signal processors configured to receive the first and second detector signals and combine the first and second detector signals into combined data prior to feeding the combined data into a single algorithm to generate a single measurement of a physiological parameter.

14. The system of claim 13, wherein the first or second detector signals comprising one or more of raw, demodulated, filtered, or converted-to-digital data.

15. The system of claim 13, wherein the one or more signal processors are further configured to map the combined data to empirical data to generate the single measurement.

16. The system of claim 13, wherein the first and second measurement sites comprise different types of body tissues.

17. The system of claim 13, wherein the first measurement site is on or around the patient's head or the patient's nose.

18. The system of claim 13, wherein the second measurement site is a peripheral site of the patient.

19. The system of claim 18, wherein the second measurement site is the patient's finger.

20. The system of claim 13, wherein the physiological parameter comprises oxygen saturation, methemoglobin, carboxyhemoglobin, total hemoglobin, and/or glucose.

21. The system of claim 13, wherein the combined data comprises one or more of a ratiometric value, non-normalized features, data from bulk absorption, or peripheral absorption signals.
